(19) United States
(12) Patent Application Publication
(10) Pub. No.: US 2012/0157515 A1
(43) Pub. Date: Jun. 21, 2012

(54) Methods and compositions for the treatment of "Burning Feet Syndrome"

(76) Inventor: John Bowman White, III, Hurricane, WV (US)

(21) Appl. No.: 12/972,499

(22) Filed: Dec. 19, 2010

(51) Int. Cl. A61K 31/385; A61K 31/198; A61P 25/00; A61K 31/197

(52) U.S. Cl. 514/440; 514/563; 514/565

(57) ABSTRACT

Disclosed herein Methods and composition for the treatment of an individual with “Burning Feet Syndrome”. Included are the manner and process of making the composition, administration, uses, and effective therapeutic amounts of Pantothenic acid and Alpha-lipoic acid with no adverse side effects. The composition can be used to prophylax (prevent), treat acute or chronic symptoms caused by “Burning Feet Syndrome”. In combination with certain adjuvants, carriers, vehicles and delivery systems to improve the efficacy.

### BACKGROUND OF THE INVENTION

1. Field of the Invention

The combination of Pantothenic acid, Alpha-lipoic acid, L-arginine are more effective in combination than one substance alone in treating symptoms of “Burning Feet Syndrome”.(4) By definition “Burning Feet Syndrome”, a neurological disorder characterized by symptoms of a burning sensation in the sole of the foot. The burning tends to be more intense at night and may also involve the hands. Possible causes include causalgia from injury to the sciatic nerve, degeneration of the spinal cord, and polyneuropathy. The condition is also associated with diabetes mellitus, kidney disease, and a B vitamin deficiency. Also known as Gopalan's Syndrome.(1)

2. Description of Related Art

Causalgia is defined as burning pain, often with trophic skin changes, due to peripheral nerve injury (i.e. Injury to sciatic nerve, degeneration of the spinal cord).(5)

Polyneuropathy is defined as a condition in which many peripheral nerves are afflicted with a disorder.(1) Therefore, “Burning Sensations” are a symptom caused by polyneuropathy disorders. These disorders may be acute or chronic.(6)

Acute polyneuropathy has many causes: infections involving a toxin produced by bacteria, as occurs in diphtheria. An autoimmune reaction as occurs in Guillain-Barre syndrome. Toxic substances, include heavy metals such as lead and mercury. Drugs, including the anticonvulsant phenytoin, some antibiotics (such as chloramphenicol, nitrofurantoin, and sulfonamides), some chemotherapy drugs (such as vinblastine and vincristine), and some sedatives (such as barbital and hexobarbital). Cancer, such as multiple myeloma, which damages nerves directly invading or putting pressure on them or by triggering an autoimmune reaction.(6)

Chronic polyneuropathy has many causes: Diabetes, Excessive use of alcohol, Nutritional deficiencies (such as thiamin deficiency), an uncommon cause in the United States, except among alcoholics who are malnourished. Anemia due to vitamin B12 deficiency (pernicious anemia), an underactive thyroid gland (hypothyroidism), liver failure, kidney failure, certain cancers, such as lung cancer. Vitamin B6 (pyridoxine) taken in excessive amounts.(6)

Specifically, Pantothenic acid deficiency in humans has been induced experimentally by administering a Pantothenic acid antagonist together with a Pantothenic acid deficient diet. Participants in this experiment complained of headache, fatigue, insomnia, intestinal disturbances, and numbness and tingling of their hands and feet.(7) Symptoms of deficiency are similar to other vitamin B deficiencies.(8) It has been noted that painful burning sensations of the feet were reported in tests conducted on volunteers. Deficiency of Pantothenic acid may explain similar sensations reported in malnourished prisoners of war.(9)

Burning is a sensation described as a paresthesia. Paresthesias are defined as abnormal sensations experienced in the absence of specific stimuli.(3) These sensations are usually described as burning, tingling or numb feelings, although they may be described as feelings of cold, warmth, prickling, pins and needles, skin crawling or itching.(3)

Burning pain in the feet has been known to occur as a distinct clinical symptom for almost two centuries. “Burning Feet Syndrome” has received scant attention in the medical literature and has been described only in anecdotal reports. There is no specific etiology and it can occur as an isolated symptom or as a part of a symptom complex in a variety of clinical settings.(10)

U.S. Pat. No. 7,803,790 to Chong, et al. the present application relates to compounds and methods for treating pain and other conditions related to TRPV3. The TRPV3 antagonist of the subject invention can be used as part of a prophylaxis or treatment for a variety of disorders and conditions which include Grierson-Gopalan syndrome (better known as burning feet syndrome). In some instances, vitamin or mineral deficiencies may lead to ulcers or other sores in the mouth. For example, deficiency in Vitamin C may lead to the oral lesions characteristic of scurvy. Deficiencies in vitamins B1, B2, B6, or B12 may also lead to oral lesions. Additionally, deficiencies in zinc, folic acid iron, selenium or calcium may lead to oral lesions. The subject TRPV3 inhibitors can be administered with vitamins and derivatives thereof including Vitamin A, Ascorbic acid (Vitamin C), alpha-tocopherol (Vitamin E), 7-dehydrocholesterol (Vitamin D), Vitamin K, Alpha-Lipoic Acid, lipid soluble anti-oxidants, and the like.

U.S. Pat. No. 7,671,061 to Morgan, et al. the present application relates to compounds and methods for treating pain, incontinence, and other conditions. The TRPV1 antagonist of the subject invention can be used as part of a prophylaxis or treatment for a variety of disorders and conditions which include Grierson-Gopalan Syndrome (better known as “Burning Feet Syndrome”). In some instances, vitamin or mineral deficiencies may lead to ulcers or other sores in the mouth. For example, deficiency in Vitamin C may lead to the oral lesions characteristic of scurvy. Deficiencies in vitamins B1, B2, B6, or B12 may also lead to oral lesions. Additionally, deficiencies in zinc, folic acid iron, selenium or calcium may lead to oral lesions. The subject TRPA 1 inhibitors can also be administered with vitamins and derivatives thereof including Vitamin A, ascorbic acid (vitamin C), alpha-tocopherol (Vitamin E), 7-dehydrocholesterol (D), Vitamin. K, Alpha-lipoic Acid, lipid soluble anti-oxidants, and the like.

U.S. Pat. No. 7,115,286 to Meredith the present disclosure concerns methods and compositions to inhibit insects from biting a subject. In preferred embodiments, the compositions may be administered orally, for example using a spray bottle to deliver to the mouth. The compositions may include one or more herbs selected from the group consisting of rice bran, peppermint, barely grass. lobelia; chlorella watercress, alfalfa, and parsley and one or more vitamins selected from the group consistent of thiamin (B1), riboflavin (B-2), niacin (B3), Pantothenic acid (B5), pyridoxine (B6), folic acid (B9), cyanocobalamin (B12), choline, inositol, d-biotin, para-amino-benzoic acid, and lecithin.

U.S. Pat. Nos. 7,569,384 and 7,521,424 Rosen et al. the present invention encompasses albumin fusion proteins. Nucleic acid molecules encoding the albumin fusion protein's of the invention are also encompassed by the invention, as are vectors containing these nucleic acid, host cell transformed with these nucleic acids vectors, and methods of making the albumin fusion proteins of the invention and using these nucleic acids, vectors, and/or host cells.

The purpose of this invention is for the prophylaxis of the basic pathophysiology or the impairment or altered nerve or nerve pathway function which causes burning feet by a disorder, within the definition of “Burning Feet Syndrome”. In the medical literature Pantothenic acid supplementation has specifically been used for “Burning Feet Syndrome”. In the medical literature alpha lipoid acid has been used as the universal antioxidant which has prophylaxed nerve tissue and help relieve burning feet (paresthesia). In the medical literature L-arginine is necessary for the production of nitric oxide within the gastrointestinal system to open sodium channels which certain vitamins like Pantothenic acid and amino acids like alpha-lipoic acid absorption within the intestine. In the medical literature L-arginine improves small vessel endothelial function in humans which improves endothelium dependent dilation and reduces monocyte and endothelial cell adhesion. Within the invention the combination additionally improves the parathesia burning of “Burning Feet Syndrome” than one lone substance.

In the United States symptomatic treatments for these parethesias include tricyclic antidepressants (TCAs), selective serotonin reuptake inhibitors, serotonin nor-epinephrine reuptake inhibitors, calcium-channel blockers, sodium channel blockers, topical capsaicin, opioids, anticonvulsants. The efficacy of a single therapeutic agent is not the rule, and simple analgesics are usually inadequate to control the paresthesia pain.(2)

Kindermann et al. in the titled article “New Drug Combination For Treating Polyneuropathy” observed that human subjects that were pretreated for symptoms of polyneuropathy with alpha-lipoic acid showed an additional improvement with a combination of Pantothenic acid. Alpha-lipoic acid was used in dosages of 300 milligrams/day and 600 milligrams/day. As Pantothenic acid was added to the combination additional improvements for the symptoms were noted in polyneuropathy (diabetic polyneuropathy, alcohol induced polyneuropathy, and unknown causes of polyneuropathy). Noted was as Pantothenic acid dose was increased to the combination the symptoms were noted to improve in 28 of 33 (84.8%) of the human subjects. Also noted was that both Alpha-lipoic acid and Pantothenic acid have a different implication in the pyruvate metabolism and therefore, this is why the combination is more effective than just one substance. At this level, the pyruvate metabolism is dependent on alpha-lipoic acid and pantothenic acid is a precursor for acetylcholine production (essential for peripheral nerve function).(4)

### Pantothenic Acid:

Gopalan C. the author of The “Burning-Feet Syndrome” and one of the persons credited for the name Grierson-Gopalan Syndrome (Burning Feet Syndrome). He was quoted in the book History of Tropical Neurology Nutritional Disorders, by Bruyn, G. W. et al. as saying “Burning feet syndrome reached epidemic proportions in 1942, likely that a lack of pantothenate (coenzyme A) was the deciding factor and that burning feet was a sympathetic neurovascular syndrome.”(11) Gopalan C. in his article The “Burning-Feet Syndrome”, noted a cure of “Burning Feet Syndrome” in 53 cases by means of parenteral calcium pantothenate after failed with thiamine, riboflavin, and nicotinic acid.(12)

New World Encyclopedia reports: pantothenic acid is essential to form coenzyme-A, production of neurotransmitter acetylcholine and thus critical in the metabolism and synthesis of carbohydrates, proteins, and fats. Pantothenic deficiencies can lead to burning feet syndrome.(13)

Free Pantothenic Acid is absorbed into intestinal cells via a saturable, sodium-dependent active transport system. At high levels of intake, when this mechanism is saturated, some pantothenic acid may also be absorbed via passive diffusion. Large doses of the vitamin, when ingested, have no reported side effects and massive doses (for example, 10 grams/day) may not yield mild intestinal distress and diarrhea at worst.(14)

Pantothenic acid is essential to normal epithelial function. Topical use of dexpanthenol (stable alcohol analog of pantothenic acid) in skin disorders has been well established. Adjunct skin care with dexpanthenol considerably improves symptoms of skin irritation, such as dryness of the skin, roughness, scaling, pruritus, erythema, erosion/fissure. Beneficial effects have been observed in patients who undergone skin transplantation or scar treatment, or therapy for burn injuries and different dermatoses.(17)

### Alpha-Lipoic Acid:

Alpha-lipoic acid is helpful in diabetic neuropathy. Alpha-lipoic acid rapidly and significantly reduces sensory symptoms and pain of diabetic neuropathy, according to the results of a double-blind trial reported in the March 2003 issue of Diabetic Care. Alpha-lipoic acid is a potent antioxidant, prevents or improves nerve conduction attributes, endothelial blood flow, and nerve Na+ K+ ATPase activity in experimental diabetes and in humans and may improve positive neuropathic sensory symptoms.(15)

Alpha-lipoic acid may be the most important antioxidant ever discovered. The only antioxidant both water and fat soluble. Alpha-lipoic acid helps regulate neuronal calcium homeostasis, regulates pro-inflammatory cytokines, and alters the expression of “toxic gene”. Therefore, Alpha-lipoic acid has been recommended as a “neuroprotector agent”.(16)

Thioctic acid (alpha-lipoic acid analog) appears to be effective in the treatment of sciatic pain caused by herniated disc and may be associated with an improvement in Neuropathy Impairment Scores in the lower limbs. This double-blind study showed the antioxidant properties of Alpha-lipoic acid helped the recovery of nerve functionality and decrease neuropathic pain.(18)

Recent studies showed that treatment with alpha-lipoic acid reduced the pain, paresthesia, and numbness in symptomatic diabetic polyneuropathy and in patients with compressive radiculopathy syndrome from disc-nerve root conflict.(19)

Alpha-lipoic acid appears to significantly improve acute microcirculation occlusion. Alpha-lipoic acid also demonstrated in patients with diabetic polyneuropathy significant improvement in microcirculation.(20)

A significant improvement with Alpha-lipoic acid for the symptomatic diabetic polyneuropathy, seen in the Total Symptom Score (TSS) within the SYDNEY 2 trial. This was noted as early as, the end of the first week, of oral therapy with Alpha-lipoic acid 1800 milligrams, and after 2 weeks at 600 milligrams and 1200 milligrams. This finding suggested that oral treatment with Alpha-lipoic acid in doses range from 600 milligrams to 1800 milligrams may be as effective as intravenous therapy using 600 milligrams/day over 3 weeks. However, because of the side effects most frequently seen with a dose-dependent increase in the incidence of nausea. In conclusion, once-daily oral treatment with 600 milligrams of Alpha-lipoic acid appears to be the most appropriate dose.(21)

### L-Arginine:

The effects of L-arginine on the physiological changes in the digestive tract associated with diabetes. The author's study concluded, thus exogenously administered L-arginine might improve the clinical manifestations of diabetes mellitus and decrease the oxidative stress in the gastrointestinal tract. In addition, the study supports the beneficial effects of L-arginine.(22)

In vivo jejunal perfusion of L-arginine, induced a dose-dependent pro-secretor effect on the jejunal transport of water, Na and Cl.(23)

L-arginine is the principal physiologic precursor of nitric oxide. L-arginine plays a role in maintaining the physiology of the gastrointestinal tract, and leads to the production of nitric oxide which affects a number of regulatory mechanisms including: vasodilation and endothelial function, neurotransmission and neuromodulation, modulation of leukocyte adhesion, insulin sensitivity inhibition of platelet aggregation, and reduction of oxidative stress.(24)

The safety of excess arginine may be affected by lysine intake as well as the total amount of protein consumed. Daily intakes of arginine and lysine from dietary protein are about 5.4 and 5.0 grams, respectively, for a person consuming 100 grams of protein. Side effects were not reported with daily doses of 1 gram L-arginine in combination with 1 gram L-ornithine given 5 days per week for 5 weeks.(26)

### Analogs, Derivatives, Synonyms

The composition of the combination within the invention will include Pantothenic acid or its analogs, or its derivatives, or its synonyms, Alpha-lipoic acid or its analogs, or its derivatives, or its synonyms, L-arginine or its analogs, or its derivatives, or its synonyms.

### METHODS OF ADMINISTRATION

The methods of administration of the combination of compounds within the invention are administered within one composition, therefore, are administered at the same time.

For administration, the combination of compounds within the invention may be made up in a solid form (e.g., capsules, tablets, granules, powders, suppositories) or liquid form (e.g., solution, suspensions, emulsions, creams, lotions, gel, with or without a patch). They may be applied in a variety of solutions and may be subject to conventional pharmaceutical operations such as sterilization and/or contain conventional adjuvants (i.e., preservatives, stabilizers, wetting agents, emulsifiers, buffers etc.).

For administration, the combination of compounds within the invention are ordinarily combined with one or more adjuvants appropriate for the indicated route of administration. For example, they may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the combination of compounds within the invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delayed material, such as glyceryl monostearate or glyceryl distearate alone or with a wax, or other materials well known in the art. Newer technology such as Bio-tract® delivery system or LiveBac® delivery system may be used in the combination of compounds within the invention.

The combination of compounds within the invention may be given by suitable route, including orally, parentally, rectally, topically in dosage unit formulations containing conventional pharmaceutically acceptable with adjuvants, carriers, vehicles and delivery systems including liposomes. The term parentally includes: subcutaneous, intravenous, intraarterial, intramuscular, intrasternal, intratendinous, intraspinal intracranial, intrathoracic, infusion techniques, intracavity, or intraperitoneally. The preferred embodiment of this combination within the invention is administered orally.

Pharmaceutically acceptable acid addition salts of the compounds suitable for the use in methods of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoracetate, propionate, caprylate, isobutyrate, oxalate, malonate, succinate, suberate, sebacate, fumarate, maleate, mandelate, benzoate, chlorabenzoate, methylbenzoate, dinitrobenzoate, phthalate, benzenesulfonate, toluenesulfonate, phenylacetate, citrate, lactate, maleate, tartrate, methanesulfonate, and the like. Also, contemplated are salts of amino acids such as arginate and the like and gluconate, galacturonate, n-methyl glutamin, etc.(25)

### Dosage Units of Each Compound of the Combination Within the Invention

Pantothenic acid and its analogs, or derivatives or synonyms

50 milligrams to 2,000 milligrams per day

Alpha-lipoic acid and its analogs, or derivatives or synonyms

25 milligrams to 600 milligrams per day

L-arginine and its analogs, or derivatives or synonyms

25 milligrams to 200 milligrams per day

Preferably the unit dosage form is prepared for twice daily administration to achieve a daily dosage of each compound within the combination of the invention. When using a delivery system (i.e., Bio-tract ®, LiveBac ®) the amounts of each compound may need to be adjusted but never exceed the milligrams per day and will be preferably administered once daily. The per dose of each compound within the combination of the invention and the dosage per day of each compound within the combination does not exceed what the medical literature sites. The milligrams per day of each compound within the combination of the invention are therapeutic in helping decrease the symptoms for the treatment of “Burning Feet Syndrome” in the medical literature. The research shows the maximum dose per day at worst may lead to intestinal distress, or nausea with no adverse side effects. The compounds within the combinations of the invention are well below the maximum dose of each compound.

### In Conclusion:

The methods of the composition within the invention shown the active ingredient Pantothenic acid has been used for the treatment of an individual with “Burning Feet Syndrome”. Throughout the medical literature the combination within the invention is more effective, than each composition alone. When using the compounds at therapeutic doses, the composition within the invention shows no adverse side effects. When using the compounds at the daily levels the composition within the invention is proven in the medical literature that the combination of the compositions can be used to prophylax and treat acute or chronic symptoms caused by “Burning Feet Syndrome”.

1. The invention relates to a combination suitable for use as a treatment of an individual with “Burning Feet Syndrome”. This neurological syndrome is characterized by burning sensations in the soles of the foot. The combination of substances in the described invention improves the burning sensation and can be favorably influenced by the described combination therapy with no adverse side effects.

2. The combination of claim 1, wherein being the main ingredients Pantothenic acid and its analogs, derivatives or synonyms, Alpha-lipoic acid and its analogs, derivatives or synonyms and L-arginine and its analogs, derivatives or synonyms.

3. The combination of claim 1, wherein methods of absorption of the combination containing conventional acceptable adjuncts, carriers, and may or may not contain time release composition or delivery system.

4. The combination of claim 1, wherein methods of administration are orally, parentally, rectally, topically.

5. The combination of claim 1, wherein methods of amounts of each compound.
